(19) United States
(12) Patent Application Publication
(10) Pub. No.: US 2010/0049243 A1
(43) Pub. Date: Feb. 25, 2010

(54) HINGED FORCEPS

(75) Inventor: Join Y. Luh, Arcata, CA (US)

Correspondence Address: MEYERTONS, HOOD, KIVLIN, KOWERT & GOETZEL, P.C., P.O. BOX 398, AUSTIN, TX, 78767-0398, US

(73) Assignee: BOARD OF REGENTS, THE UNIVERSITY OF TEXAS SYSTEM, Austin, TX (US)

(21) Appl. No.: 12/226,301

(22) PCT Filed: Apr. 11, 2007
(86) PCT No.: PCT/US2007/066427

§ 371 Date: Oct. 23, 2009

Related U.S. Application Data
(60) Provisional application No. 60/790,916, filed on Apr. 11, 2006.

(51) Int. Cl. A61B 17/28

(52) U.S. Cl. 606/208

(57) ABSTRACT

In some embodiments, a forceps may include at least two arms, at least one finger grip, and a hinge. Finger grips may be positioned on an arm. A hinge may couple two or more arms together In an embodiment, a hinge may allow 60 degrees of separation between coupled arms. A hinge may allow uninhibited movement of one arm relative to the other arm.

### BACKGROUND

1. Field of the Invention

The present invention relates to forceps. More particularly, the invention relates to forceps that include finger grips and/or hinges.

2. Brief Description of the Related Art

Forceps are commonly used in medical procedures, engineering applications, food-service applications, and beauty applications. Forceps usually include two arms fused together at an end or joined with a spring mechanism. The fused joint or spring mechanism causes inhibited motion of the arms of the forceps. In such instances, movement of the arms with respect to each other is resisted until a force is applied to the forceps to overcome the resistive force of the spring mechanism or fused joint. Thus, a user must constantly apply pressure to the arms to retain the altered position of the arms with respect to each other. In addition, a user must maintain pressure on the forceps to control the forceps while applying pressure to the forceps to retain an object. With repetitive use, this need to apply pressure to the arms will cause fatigue. A user often must also worry about dropping the forceps during use. For example, during long surgeries, doctors may experience hand strain that may cause loss of control of the forceps. This even occurs in the so-called “reverse” forceps where applying pressure opens the forceps. In the beauty industry, cosmetologists who use forceps to remove hair also may experience hand fatigue since they have to repetitively apply pressure to the arms of the forceps during use. In the food industry, tongs used during cooking or in buffet lines may result in hand fatigue from repetitive application of pressure by the hand being positioned too close to the tongs when picking up food using the tongs. Customers with arthritis often struggle with the tongs provided in restaurants (e.g., such as at serving buffets).

Currently there exists a need for more comfortable forceps that reduce at least some of the pressures needed to use the forceps.

### SUMMARY OF THE INVENTION

In an embodiment, forceps may include at least two arms, one or more finger grips, and a hinge coupling the two arms to each other. At least one finger grip may be positioned on at least one of the arms. The hinge may be positioned at an end of each of the arms. The hinge may restrict the range of motion of the arms with respect to each other. The hinge may function to allow a predetermined separation between the arms of the forceps. In some embodiments, the hinge may allow at least 30 degrees, 45 degrees, 60 degrees, at least 90 degrees or at least 180 degrees of separation between the arms. In certain embodiments, the hinge may allow substantially uninhibited movement of one of the arms with respect to the other arms. In an embodiment, the forceps may be formed of a material capable of being sterilized. In some embodiments, forceps may be formed of a disposable material. An embodiment may also be applied to a reverse forceps design.

### BRIEF DESCRIPTION OF THE DRAWINGS

Features and advantages of the methods and apparatus of the present invention will be more fully appreciated by reference to the following detailed description of presently preferred but nonetheless illustrative embodiments in accordance with the present invention when taken in conjunction with the accompanying drawings in which:

FIG. 1 depicts an embodiment of a pair of forceps.

FIG. 2A depicts an embodiment of a pair of forceps in a partially open position.

FIG. 2B depicts an embodiment of a pair of forceps opened such that the arms are separated by 180 degrees.

FIG. 3 depicts an embodiment of forceps with three finger grips.

FIG. 4A depicts an embodiment of a pair of forceps with flexible finger grips.

FIG. 4B depicts an embodiment of forceps with circular finger grips.

FIG. 5 depicts an embodiment of a pair of forceps for use as an eating utensil.

While the invention is susceptible to various modifications and alternative forms, specific embodiments thereof are shown by way of example in the drawings and will herein be described in detail. The drawings may not be to scale. It should be understood that the drawings and detailed description thereto are not intended to limit the invention to the particular form disclosed, but to the contrary, the intention is to cover all modifications, equivalents and alternatives falling within the spirit and scope of the present invention as defined by the appended claims.

### DETAILED DESCRIPTION OF EMBODIMENTS

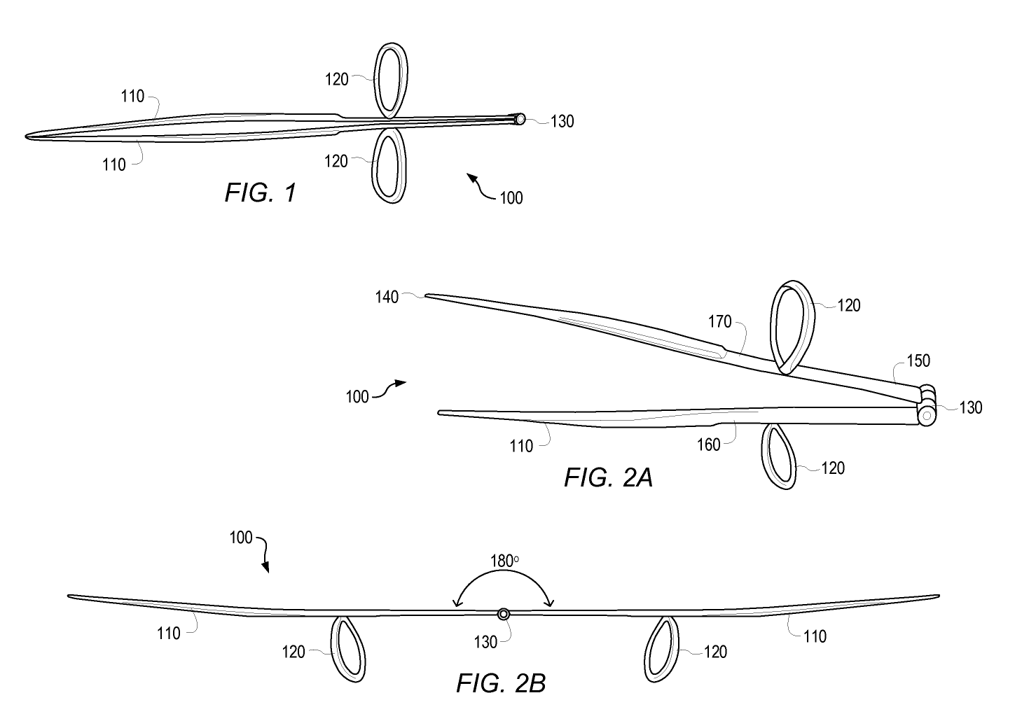

In various embodiments, forceps 100 includes arms 110, one or more finger grips 120, and a hinge 130, as depicted in FIG. 1. Forceps may be used to hold objects, close areas, and/or open areas. For example, during surgery, forceps may be used to hold a blood vessel, an organ, or tissue; clamp an area, an organ, or a blood vessel closed; or open an area for surgery. Forceps, such as tweezers, may be used in the beauty industry to remove hair. Forceps, such as tongs, may be used in the food service industry to allow a user to hold and transfer food. Forceps, such as chopsticks, may be used during cooking and/or dining.

Forceps may be formed from a variety of metal and/or non-metal materials. In an embodiment, forceps may be formed of non-metal materials such as plastic, wood, rubber, ivory, or combinations thereof. Forceps may be formed such that they are disposable. Forceps may be formed from one or more materials that may be sterilized. Forceps may be formed from stainless steel or another autoclaveable material. Forceps may be formed of a food-safe material.

In some embodiments, forceps 100 may include two or more arms 110. In some embodiments, forceps 100 may include three or more arms 110. In some embodiments, forceps 100 may include four or more arms 110. An arm 110 may be an elongated member or a conduit. An arm 110 may have a substantially square, rectangular, circular, oval, oblong, or irregular cross-sectional area. The length of an arm 110 may be selected based on the application of the forceps 100. For example, arms 110 in forceps 100 for clamping a region of the body may be larger than arms in forceps for closing a capillary. In an embodiment, at least a portion of an arm 110 may taper. For example, an arm 110 may taper away from hinge 130 and decrease in width. In another embodiment, an arm 110 may taper proximate an end of the arm.

An arm 110 may have an end 140 that is free and an end 150 that is coupled to another arm via a hinge 130, as depicted in FIG. 2A. An end 140 of an arm 110 may have a shape designed to facilitate gripping. For example, an end 140 of an arm 110 may be serrated. An end 140 of an arm 110 or an area proximate an end of an arm may be texturized to facilitate gripping. In an embodiment, an interior surface 160 of the arm may be texturized. An end 140 of an arm 110, an area proximate an end of an arm, and/or an interior surface 160 of the arm may include protrusions, ribs, a cross-hatch pattern, or other texture to facilitate gripping.

In some embodiments, a portion of an end 140 of an arm 110 may be formed of and/or coated with a material configured to facilitate gripping. For example, forceps 100 may include a rubber tip at an end 140 of each arm 110. In certain embodiments, at least a portion of an end 140 of an arm 110 may be formed of and/or coated with a material configured to inhibit damage to objects being grasped.

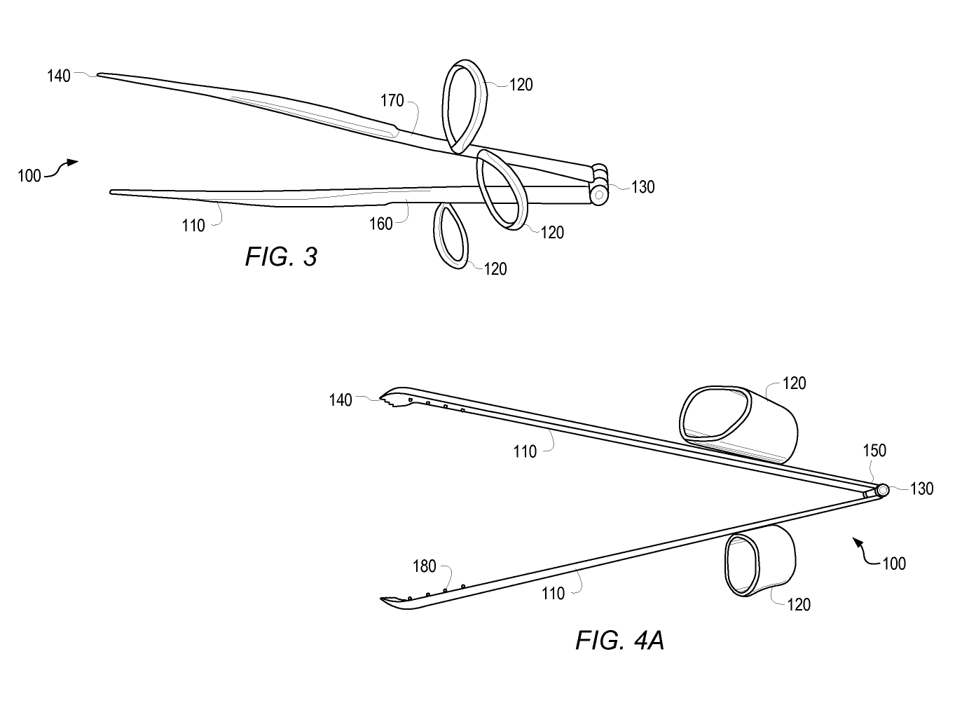

At least one finger grip 120 may be positioned on an arm 110. A finger grip 120 may be positioned on each arm 110. In an embodiment, two or more finger grips 120 may be positioned on each arm 110, see FIG. 3. For example, forceps 100 may include at least two finger grips 120 on one arm 110 to accommodate a user's index finger and middle finger during use and one finger grip on another arm to accommodate a user's thumb. When more than one finger grip 120 is positioned on an arm 110, it may be desirable to offset the finger grips from one another. In an embodiment, a first finger grip 120 may be positioned on an outer surface 170 and a second finger grip 120 may be positioned on or near an edge of the arm 110 between the outer and inner surface 160. It also may be desirable, when forceps 100 includes more than one finger grip 120 on an arm 110, to position a first finger grip at a different distance from the hinge 130 than the second finger grip on the same arm. Although using two finger grips on an arm is described above, an arm may include more than two finger grips in other embodiments.

In some embodiments, the position of finger grips 120 on an arm 110 may be adjustable or fixed. Finger grips 120 may be positioned on approximately the middle third of the length of the forceps. Finger grip 120 may be positioned proximate a center of an arm 110 and/or on an exterior surface 170 of an arm. It may be advantageous to position the finger grips 120 on exterior surface 170 of an arm 110 to increase control of forceps 100. Finger grips 120 may be positioned at approximately the same position on each arm 110 of forceps 100 or at different positions. Finger grips 120 may be positioned on arms 110 of a forceps 100 so that hinge 130 of the forceps contacts the user's palm during use. Finger grips 120 may be positioned on the arms 110 of a forceps 100 so that the hinge 130 of the forceps 100 does not contact the user's palm during use. Finger grips 120 may be positioned on an arm 110 at a distance from an end 140 of the arm so that a user may comfortably hold the forceps in the user's hand while maintaining the desired control of the forceps.

In some embodiments, one or more finger grips 120 may be positioned approximately perpendicular to or oblique to a surface of the arm 110. A finger grip 120 may be positioned obliquely relative to a surface of an arm 110, as depicted in FIGS. 2A and 2B. Positioning a finger grip 120 obliquely to a surface of arm 110 may increase user comfort. For example, during surgery, a user may find it more comfortable to control and/or retain arms 110 of forceps 100 using finger grips 120 rather than by contacting the arms with the user's fingertips.

The size and/or shape of a finger grip may vary. A finger grip may be large enough for one or more of a user's fingers to be placed at least partially in the finger grip. In an embodiment, a finger grip may only be large enough for the user to position one finger at least partially in the finger grip. A finger grip may have a circular, semi-circular, oval, oblong, square, rectangular, or an irregular shape. A finger grip may be in the shape of a ring, c-shaped ring, an oval-shaped ring, or thimble shaped. A finger grip may be designed such that a user may be able to position a finger through an opening in the finger grip. The opening of the finger grip may be sized so that only a portion of a user's finger may pass through an opening. In an embodiment, a finger grip may cover at least a portion of an end of a user's finger. A finger grip may allow a user to position a tip of a finger in the opening of the finger grip. The finger grips may be flexible. For example, the finger grips may approximately or at least partially conform to the shape of a user's finger. In an embodiment, the finger grips may be similar to a tip of a surgical glove.

In an embodiment, a finger grip may be formed from a material similar to or different from the arms and/or hinge of the forceps. The finger grips may be formed of a plastic, such as latex, rubber, and/or metal, such as stainless steel. The finger grips may be formed of a material that is configured to be disposed of after one use. The finger grips may be formed of a flexible material. The finger grips may be formed of any material capable of being sterilized and/or autoclaved. The finger grips may be formed of a food-safe material.

A finger grip may facilitate movement and/or control of an arm of a forceps by a user. It may be desirable to include finger grips on a forceps to reduce strain on a user's hand. During use, a user may only need to apply pressure to the forceps to grip an object and not to retain the forceps in the user's hand since the finger grips would inhibit the forceps from being dropped by the user.

A hinge 130 may couple two or more arms 110 of forceps 100 together, see FIGS. 2A and 2B. In certain embodiments, hinge 130 may be positioned at an end 150 of each of the arms 110 of forceps 100. In an embodiment, hinge 130 may be a pin in an opening at an end 150 of each arm 110. The hinge may include a ball bearing. The hinge 130 may allow no greater than 30 degrees of separation, at least 30 degrees of separation, at least 45 degrees of separation, at least 60 degrees of separation, at least 75 degrees of separation, at least 90 degrees of separation, at least 135 degrees of separation, or at least 180 degrees of separation between coupled arms 110. Hinge 130 may allow substantially uninhibited movement of at least one of the arms 110 relative to the other arm. Utilizing forceps with a hinge capable of such uninhibited movement may be advantageous when compared with forceps with fused or spring loaded hinges. Fused or spring loaded hinges typically require a substantial force from a user to alter the position of one arm relative to the other arm. The described embodiments of forceps have uninhibited movement that is controlled by a user through use of the finger grips. A user will typically experiences less hand fatigue and/or strain during prolonged or repetitive use of such a forceps since less force is required to move the arm of the forceps.

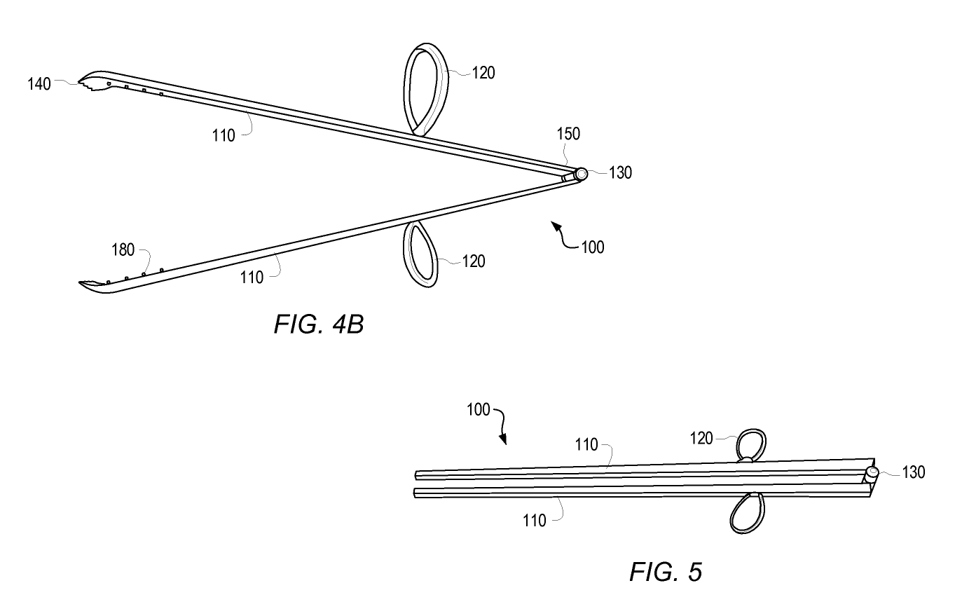

In some embodiments, the forceps may be designed for specific uses, such as medical, food service, eating, cooking, semiconductor fabrication, or small-scale laboratory experiments. FIGS. 4A and 4B depict embodiments of forceps 100 designed for food service applications. Forceps 100 may include tong-like arms 110. Finger grips 120 may be positioned on arms 110. Finger grip 120 may be positioned on arm 110 at a distance from an end 140 of the arm so that a user may comfortably hold the forceps in the user's hand. Finger grips 120 may be conduits and/or made from a flexible material, see FIG. 4A. In an embodiment, finger grips may be circular and resist deformation, see FIG. 4B. An arm 110 of forceps 100 may be serrated at end 140, as depicted in FIGS. 4A and 4B. End 140 of arm 110 of forceps 100 may include protrusions 180. Protrusions 180 may facilitate retaining food in ends 140 of forceps 100. Hinge 130 of forceps 100 may allow arms 110 to be separated by no greater than 30 degrees of separation, at least 30 degrees of separation, at least 45 degrees, at least 60 degrees, at least 75 degrees, at least 90 degrees, at least 135 degrees, or at least 180 degrees. The hinge may function to allow a predetermined separation between the arms of the forceps. Allowing a large separation of the arms may allow a user to grab large objects with the forceps. For example, a user may be able to grab large chicken breasts, ribs, and other food that may not be easily grasped with currently available commercial tongs. In addition, using forceps 100 with hinge 130 allows a user to grab small and large objects with the same forceps. For example, a restaurant may use a single forceps that allows enough separation between arms of the forceps for a user to grab a large chicken breast and a small enough separation between arms for a user to grab a small chicken wing with the forceps.

In another embodiment, forceps 100 may be an eating and/or cooking utensil, such as a chopstick, as depicted in FIG. 5. The forceps 100 may be formed from a food-safe material. Arms 110 of the forceps 100 may have a substantially square cross-section. Arms 110 of forceps 100 may taper. At least one finger grip 120 may be positioned on each arm 110. Finger grip 120 may be positioned at a distance from hinge 130 such that a user may comfortably hold the chopstick-like forceps. It may be easier for a user to eat and/or cook with forceps 100 with finger grips 120 than currently available chopsticks since the forceps are easier to operate with the finger grips.

It is to be understood that the terminology used herein is for the purpose of describing particular embodiments only, and is not intended to be limiting. As used in this specification, the singular forms “a”, “an” and “the” include plural referents unless the content clearly indicates otherwise. Thus, for example, reference to “a hinge” includes a combination of two or more hinges and reference to “a plastic” includes mixtures of plastics.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A forceps comprising:
at least two arms;
at least one finger grip positioned on at least one of the arms; and
a hinge coupling the two arms together, wherein the hinge is configured to allow a predetermined separation between the two arms.

2. The forceps of claim 1, wherein at least one finger grip is positioned on each of the arms.

3. The forceps of claim 1, wherein at least one finger grip is positioned proximate a center of an arm.

4. The forceps of claim 1, wherein at least one finger grip is positioned on an exterior side of the arm.

5. The forceps of claim 1, wherein at least one finger grip comprises a c-shaped ring.

6. The forceps of claim 1, wherein at least one finger grip comprises a ring.

7. The forceps of claim 1, wherein at least one finger grip comprises an oval shaped ring.

8. The forceps of claim 1, wherein at least one finger grip is configured to cover at least a portion of an end of a user's finger.

9. The forceps of claim 8, wherein at least one finger grip is thimble shaped.

10. The forceps of claim 1, wherein at least one finger grip is positioned obliquely relative to a surface of the arm.

11. The forceps of claim 1, wherein the hinge is positioned at an end of each of the arms.

12. The forceps of claim 1, wherein the hinge comprises a pin positioned through an opening at an end of each of the arms.

13. The forceps of claim 1, wherein the forceps are capable of being sterilized.

14. The forceps of claim 1, wherein the forceps are capable of being autoclaved.

15. The forceps of claim 1, wherein the hinge is configured to allow a separation of approximately 30 degrees or less between the two arms.

16. The forceps of claim 1, wherein the hinge is configured to allow a separation of at least approximately 30 degrees, at least approximately 45 degrees, at least approximately 60 degrees, at least approximately 75 degrees, at least approximately 90 degrees, at least approximately 135 degrees, or at least approximately 180 degrees between the two arms.

17. The forceps of claim 1, wherein the hinge is configured to allow substantially uninhibited movement of one of the arms relative to the other arm.

18. The forceps of claim 1, further comprising at least three arms.

19. The forceps of claim 1, further comprising at least four arms.

20. A method of grasping an object comprising using a forceps, wherein the forceps comprise:
at least two arms;
at least one finger grip positioned on at least one of the arms; and
a hinge coupling the two arms together, wherein the hinge is configured to allow a predetermined separation between the two arms.

21. A forceps comprising:
at least two arms; and
a hinge coupling the two arms together, wherein the hinge is positioned at an end of each of the two arms, and wherein the hinge is capable of allowing at least approximately 60 degrees of separation between the two arms.

22. A forceps comprising:
at least two or more arms;
at least one finger grip positioned on at least one of the arms; and
a hinge coupling at least two of the arms together, wherein the hinge is capable of allowing at least approximately 60 degrees of separation.

23. A forceps comprising, at least two arms coupled by a hinge.
